(19) United States
(12) Patent Application Publication
(10) Pub. No.: US 2016/0340327 A1
(43) Pub. Date: Nov. 24, 2016

(54) PROCESS FOR PREPARING AMORPHOUS CABAZITAXEL

(71) Applicant: SHILPA MEDICARE LIMITED, RAICHUR (IN)

(72) Inventors: SRIRAM RAMPALLI, VIZIANAGARAM (IN); PRASHANT PUROHIT, RAICHUR (IN); PRADEEP POTHANA, VIZIANAGARAM (IN); ANAND KUMAR DUNGA, VIZIANAGARAM (IN); AKSHAY KANT CHATURVEDI, RAICHUR (IN)

(73) Assignee: SHILPA MEDICARE LIMITED, RAICHUR (IN)

(21) Appl. No.: 15/186,432

(22) Filed: Jun. 18, 2016

Related U.S. Application Data
(63) Continuation of application No. 14/650,296, filed on Jun. 6, 2015.

(30) Foreign Application Priority Data
Dec. 31, 2012 (IN) 5516/CHE/2012
Sep. 4, 2013 (IN) 3957/CHE/2013

(51) Int. Cl. C07D 305/14

(52) U.S. Cl. CPC C07D 305/14

(57) ABSTRACT

The present invention provides a process for preparing amorphous Cabazitaxel from the solvate form of Cabazitaxel.

The present invention also provides novel diisopropyl ether solvate form of Cabazitaxel (I),

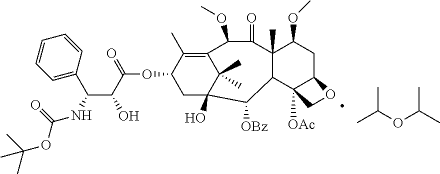

and process for preparation thereof.

Said amorphous Cabazitaxel and crystalline diisopropyl ether solvate of Cabazitaxel of the present invention can be utilized in preparing the pharmaceutical composition/s useful in the treatment of cancer.

### RELATED APPLICATIONS

This application is a continuation of U.S. patent application Ser. No. 14/650,296, filed Jun. 6, 2015, now US Patent Publication. No. 2015-0315164 A1, this claims priority under 35 U.S.C. Section 371 to PCT Patent Application No. PCT/IB2013/061089, filed Dec. 18, 2013, which claims priority to IN Patent Application No. 3597/CHE/2013, filed on Sep. 4, 2013 and 5516/CHE/2012, filed Dec. 31, 2012, the disclosures of each of which are incorporated by reference herein in their entirety.

### FIELD OF THE INVENTION

The present invention provides a process for preparing amorphous Cabazitaxel from the solvate faun of Cabazitaxel selected from diisopropyl ether solvate, ethyl acetate solvate, acetone solvate, ethanol solvate, monohydrate or dihydrate form of Cabazitaxel.

The present invention further relates to diisopropyl ether solvate form of Cabazitaxel (I),

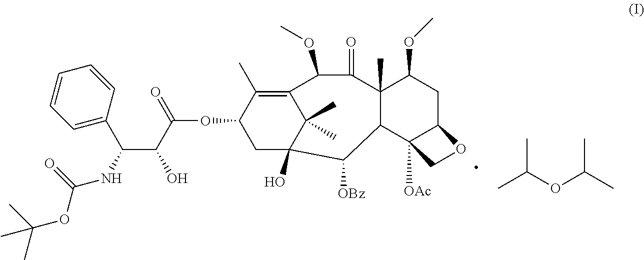

and process for preparation thereof.

Also this application presents pharmaceutical composition comprising crystalline diisopropyl ether solvate of Cabazitaxel or amorphous Cabazitaxel as an active pharmaceutical ingredient, wherein the pharmaceutical composition is having anti-cancer activity.

### BACKGROUND OF THE INVENTION

(2α,5β,7β,10β,13α)-4-acetoxy-13-({(2R,3S)-3[(tert-butoxycarbonyl) amino]-2-hydroxy-3-phenylpropanoyl}oxy)-1-hydroxy-7,10-dimethoxy-9-oxo-5,20-epoxytax-11-en-2-yl benzoate is the chemical name of Cabazitaxel (Ia)

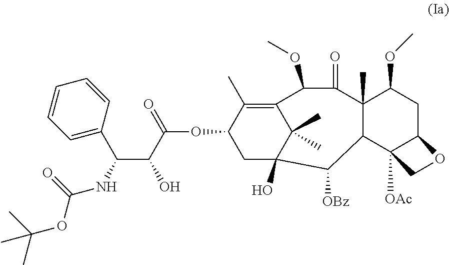

Cabazitaxel as its acetone solvate was approved by USFDA as JEVTANA™ and is chemically mentioned in the label as (2α,5β,7β,10β,13α)-4-acetoxy-13-({(2R,3S)-3[(tert-butoxy-carbonyl) amino]-2-hydroxy-3-phenylpropanoyl} oxy)-1-hydroxy-7,10-dimethoxy-9-oxo-5,20-epoxytax-11-en-2-yl benzoate-propan-2-one (1:1) solvatomorph (Cabazitaxel : acetone). Cabazitaxel is a white to off-white powder and is lipophilic in nature, practically insoluble in water.

Bouchard et al in U.S. Pat. No. 5,847,170 provided the first disclosure of (2α,5β,7β,10β,13α)-4-acetoxy-13-({(2R,3S)-3[(tertbutoxycarbonyl) amino] -2-hydroxy-3-phenylpropanoyl} oxy)-1-hydroxy-7,10-dimethoxy-9-oxo-5,20-epoxytax-11-en-2-yl benzoate (also known as Cabazitaxel), also describing the process for preparing Cabazitaxel. Kung Liang-Rem et al in US2012149925A1 disclosed a process for preparing Cabazitaxel by reacting beta-lactam side chain with a protected baccatin derivative in the presence of one or more Lewis acids and a base agent, wherein Lewis acid may be selected from LiBr, MgBr₂, CsBr, ZnBr₂, ZnCl₂, CuBr, Cu(CF₃SO₄)₂, BF₃.OEt₂, KBr, TiCl₄, SnCl₂, ScCl₃, VCl₃, AlCl, InCl₃, Al₂CO₃, CeCl₃, Ag₂O, ZnClO₄, LiClO₄, Ti{OCH(CH₃)₂}₄ or any combination thereof.

The Cabazitaxel form mentioned in the USFDA label as (2α,5β,7β,10β,13α)-4-acetoxy-13-({(2R,3S)-3 [(tert-butoxy-carbonyl) amino]-2-hydroxy-3-phenylpropanoyl}oxy)-1-hydroxy-7,10-dimethoxy-9-oxo-5,20-epoxytax-11-en-2-yl benzoate-propan-2-one (1:1) solvatomorph (Cabazitaxel: acetone) is designated as Form-A in EMEA scientific discussion as well as in the subsequent patent US7241907, which also describes its process for preparation by crystallization from an aqueous/acetone solution.

It appears from the literature that in order to achieve therapeutic role, it is Cabazitaxel base molecule that plays the active role and not necessarily its solvates as long as stable solvate or base form is known to exist. In line with this, US 20120301425A1 (Eq. WO 2011051894A1) on page 2 mentions that-‘Cabazitaxel may be administered in base form, or in the form of a hydrate. It may also be a solvate, i.e. a molecular complex characterized by the incorporation of the crystallization solvent into the crystal of the molecule of the active principle (see in this respect page 1276 of J. Pharm. Sci. 1975, 64(8), 1269-1288). In particular, it may be an acetone solvate, and, more particularly, may be the solvate described in WO 2005/02846. It may be an acetone solvate of Cabazitaxel containing between 5% and 8% and preferably between 5% and 7% by weight of acetone (% means content of acetone/content of acetone+cabazitaxel χ100). An average value of the acetone content is 7%, which approximately represents the acetone stoichiometry, which is 6.5% for a solvate containing one molecule of acetone.’

Billot Pascal et al in U.S. Pat. No. 8,378,128 appear to cover many crystalline forms, which includes crystalline forms as anhydrides, solvates and ethanol hetero-solvates and hydrates forms of Cabazitaxel. The disclosure in this application provides nearly 11 new polymorphic forms-which include five (5) new crystal forms of Cabazitaxel anhydrous material designated as Form B, C, D, E, F, which are characterized by - Form B- DSC MP. 150° C., Form C- DSC MP. 146° C., Form D- DSC MP. 175° C., Form E- DSC MP. 157° C. and Form F- DSC MR 148° C. along with their characteristic XRPD pattern. It also disclosed four new crystal forms of ethanolate and heterosolvate of Cabazitaxel designated as Form 13 Ethanolate, Form D Ethanolate, Form E Ethanolate, and Form F Ethanolate/ Water heterosolvate. Two hydrate forms of Cabazitaxel i.e. Form C as Monohydrate and Form C as Dihydrate are also part of the disclosure. This patent specification also mentioned that only Form D anhydrous is highly stable even more stable that acetone solvate form (Form A).

Simo et al in W02012142117 Al also disclosed different polymorphic forms of Cabazitaxel- Form I i.e. toluene solvate, Form II i.e. methyl tert-butyl ether (“MTBE”) solvate, Form III i.e. 2-propanol solvate, Form IV i.e. n-butanol solvate and Form V i.e. 1-propanol solvate.

Recently Henschke et al in US2013065955 Al provided several crystalline forms of Cabazitaxel designated as Form Cl, C2, C3, C4, C5, C6, C7, C8, C9, C8b and C9p. This patent application also disclosed that crystalline Form Cl is an anhydrous isopropanol solvate of Cabazitaxel, Form C8b is a monohydrate DMSO solvate and crystalline Form C9p is a monohydrate acetic acid solvate of Cabazitaxel.

Further, crystalline polymorphs of Cabazitaxel have been disclosed by Lahiri et al in WO2013080217 A2. The different polymorphs disclosed have been referred to as Form-1, Form-2, Form-3, Form-4, Form-5, Form-6, Form-7, Form-8, Form-9, Form-10, Form-11, Form-12, and Form-13.

Didier et al in WO2013088335 Al have provided crystalline ethyl acetate solvate form of the 4α-acetoxy-2a-benzoyloxy-5β, 20-epoxy-1β-hydroxy-7β,10β-dimethoxy-9-oxo-11-taxen-13α-yl(2R,3S)-3-tert-butoxycarbonylamino-2-hydroxy-3-phenylpropionate, which is defined as Form A.

Also amorphous form of Cabazitaxel has been disclosed in some recent applications for e.g. W02012142117 A1 which provides a powdery, non-foamy form of Cabazitaxel, WO2013111157 A1 which provides a non-solvated amorphous four of Cabazitaxel and WO2013065070 A1.

Existence of polymorphism is known to be a unique phenomenon in solid materials, wherein existence of different physical forms including shape, size, and arrangement of molecules in the physical state or polymorphs of same compound are known in the nature. A single compound, or a salt complex, may give rise to a variety of solids having distinct physical properties, which may result in substantial differences in bioavailability, stability, and other differences between production lots of formulated pharmaceutical products. Due to this reason, since polymorphic forms can vary in their chemical and physical properties, Regulatory Authorities often require that efforts to be made to identify all stable polymorphic forms e.g., hydrate or anhydrate, crystalline or amorphous, solvated or un-solvated forms, etc. of the drug substances. However, the existence and possible numbers of polymorphic forms for a given compound cannot be predicted. It is imperative that- adequate scientific diligence/efforts are required to explore new salts/solvates and other polymorphs for the same compound already known to exist in nature.

There are no “standard” procedures known to exists that can be used to prepare polymorphic forms of a substance. Hence, besides identification of the different polymorphic foinis of a compound, concern has always remained for the process of preparation of these polymorphic forms. Inventors of the present application thus provide a process for preparing amorphous Cabazitaxel from the solvate forms of Cabazitaxel, wherein the process appears to be highly efficient, cost-effective, industrially amenable to scale up, solves purity/ compliance related issues and overcomes the drawbacks of various prior disclosed processes, e.g., multiple solvent combinations as well as multiple steps.

Exploring novel solvate /polymorphic forms of pharmaceutically active / useful compounds always provide a further opportunity to improve the drug performance characteristics of such product.

In continuation of our endeavor to find new solvate /polymorphic forms of Cabazitaxel, inventors of the present application provide a new stable solvate form of Cabazitaxel, which is diisopropyl ether solvate of Cabazitaxel. Diisopropyl ether solvate of Cabazitaxel is very stable and retains its characteristic properties on long term storage also, wherein the physicochemical properties remain substantially the same. This stable form further offers various advantages in terms of favorable impurity profile, flowability, solubility, morphology, crystal habit, minimal polymorphic conversion, low hygroscopicity, low content of residual solvents and ease of formulation.

### SUMMARY OF INVENTION

Particular aspects of the present specification relate to a process for preparing amorphous Cabazitaxel from the solvate form of Cabazitaxel, comprising the steps of
i. providing a solution of Cabazitaxel solvate form with solvent methanol or 2-methoxyethanol;
ii. maintaining the reaction mass under stirring to dissolve completely at temperature ranging from 10-35° C.;
iii. heating the solution up to the temperature ranging from 40-60° C.;
iv. optionally holding the solution under stirring for at least 20 minutes but not more than 60 minutes;
v. removing the solvent by distillation and isolating the amorphous Cabazitaxel.
   In a preferred aspect of the present application the solvate form of Cabazitaxel used to prepare amorphous Cabazitaxel, is Cabazitaxel diisopropyl ether solvate form.

Further aspects of the present specification relate to the novel solvate form of Cabazitaxel which is Cabazitaxel diisopropyl ether solvate (I), and process for its preparation.

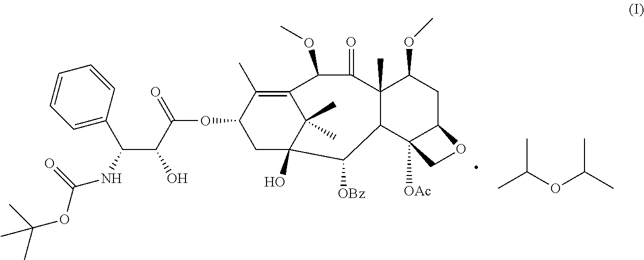

Cabazitaxel diisopropyl ether solvate (I) obtained by the process according to the present invention is found to exist as a crystalline form, designated as Form-SD.

In one aspect of the present application, the present invention provides diisopropyl ether solvate of Cabazitaxel (Form-SD), wherein the diisopropyl ether solvate of Cabazitaxel exists as a crystalline form designated as Form-SD, which is characterized by X-ray powder diffraction pattern comprising at least 8 characteristic diffraction angle peaks selected from the XRPD peak set of 7.5, 7.7, 8.6, 13.5, 14.2, 15.0, 17.5, 19.9, 21.7, 21.8, 22.9 and 23.4±0.2 2θ° and DSC isotherm comprising at least one endothermic peak ranging between 145 to 160° C.

In another aspect of the present application, diisopropyl ether solvate of Cabazitaxel (I) obtained as crystalline Form-SD, is further characterized by IR absorption spectrum having characteristic peaks expressed in cm⁻¹ at approximately 3542 cm⁻¹, 3376 cm¹, 2977 cm⁻¹, 2823 cm⁻¹, 1721 cm⁻¹, 1601 cm⁻, 1520 cm⁻¹, 1169 cm⁻¹ and 848 cm⁻¹.

In a further aspect of the present application, it relates to diisopropyl ether solvate of Cabazitaxel (I) obtained as crystalline Form-SD, and is characterized by X-ray powder diffraction pattern substantially according to FIG. 2, DSC isothermal pattern substantially according to FIG. 3 and IR absorption spectrum substantially according to FIG. 4.

In still further aspect of the present invention, diisopropyl ether solvate of Cabazitaxel (I) obtained as crystalline Form-SD, is characterized by diisopropyl ether content ranging from 8-12% w/w.

In yet another aspect of the present application, it relates to process for preparing diisopropyl ether solvate of Cabazitaxel (Form-SD), comprising the steps of:
a) Providing a solution of Cabazitaxel in an organic solvent;
b) Adding diisopropyl ether to the reaction mixture;
c) Subjecting the reaction mixture to continuous stirring at temperature ranging between 25 to 55° C.;
d) Filtering the solid material from reaction mass and washing it with diisopropyl ether;
e) Optionally repeating the steps a) to d);
f) Isolating crystalline diisopropyl ether solvate of Cabazitaxel (Form-SD).

In a further aspect of the present invention, it relates to a pharmaceutical composition comprising crystalline diisopropyl ether solvate of Cabazitaxel or amorphous Cabazitaxel as an active pharmaceutical ingredient, together with one or more pharmaceutically acceptable excipients, wherein the said pharmaceutical composition has therapeutic usefulness because of its anti-cancer activity.

Further particular aspects of the invention are detailed in the description of invention, wherever appropriate.

### BRIEF DESCRIPTION OF THE DRAWINGS

FIG. 1 is an example of X-ray powder diffraction (“XRPD”) pattern of Amorphous Cabazitaxel obtained according to the process of the present invention.

FIG. 2 is an example of X-ray powder diffraction (“XRPD”) pattern of diisopropyl ether solvate of Cabazitaxel (I) crystalline Form-SD.

FIG. 3 is an example of differential scanning calorimetry (“DSC”) curve of diisopropyl ether solvate of Cabazitaxel (I) crystalline Form-SD.

FIG. 4 is an example of IR spectral pattern of diisopropyl ether solvate of Cabazitaxel (I) crystalline Form-SD.

FIG. 5 is an example of ¹H NMR spectrum of diisopropyl ether solvate of Cabazitaxel (I).

FIG. 6 is an example of Mass spectrum of diisopropyl ether solvate of Cabazitaxel (I).

FIG. 7 is an example of Thermo Gravimetric Analysis (“TGA”) pattern of diisopropyl ether solvate of Cabazitaxel (I) crystalline Form-SD.

### ABBREVIATIONS

| | |
|---|---|
| ¹H NMR | Proton Nuclear Magnetic Resonance |
| DSC | Differential Scanning Calorimetry |
| DMSO | DiMethylSulfOxide |
| DMF | DiMethylFormamide |
| HPLC | High Performance Liquid Chormatography |
| IR | Infrared Spectroscopy |
| MP | Melting Point |
| TGA | Thermo Gravimetric Analysis |
| THF | TetraHydroFuran |
| XRPD | X-Ray Powder Diffraction |

### DETAILED DESCRIPTION

As set forth herein, embodiments of the present invention relate to a process for preparing amorphous Cabazitaxel from the solvate form of Cabazitaxel, comprising the steps of
i. providing a solution of Cabazitaxel solvate form with solvent methanol or 2-methoxyethanol;
ii. maintaining the reaction mass under stirring to dissolve completely at temperature ranging from 10-35° C.;
iii. heating the solution up to the temperature ranging from 40-60° C.;
iv. optionally holding the solution under stirring for at least 20 minutes but not more than 60 minutes;
v. removing the solvent by distillation and isolating the amorphous Cabazitaxel.

A solution of crystalline Cabazitaxel solvate form in solvent methanol or 2-methoxyethanol is provided at temperature ranging between 20-30° C. The reaction mixture obtained is maintained under stirring at a temperature ranging from 10-35° C. In one of the particular embodiment, stirring of the reaction mass was done at temperature of 25-30° C. Stirring of the reaction mass is done for the time duration ranging between 10 to 60 minutes; however, depending upon the progress of the reaction-it may extend in order to achieve the complete dissolution.

The temperature of the reaction solution obtained above is raised to 40-60° C. In a particular embodiment, the temperature of reaction solution obtained was raised to 50-55° C. The solution is optionally maintained for time duration of 20-60 minutes at the raised temperature of 40-60° C. The time duration of stirring depends upon the progress of the reaction, which is checked intermittently during the reaction.

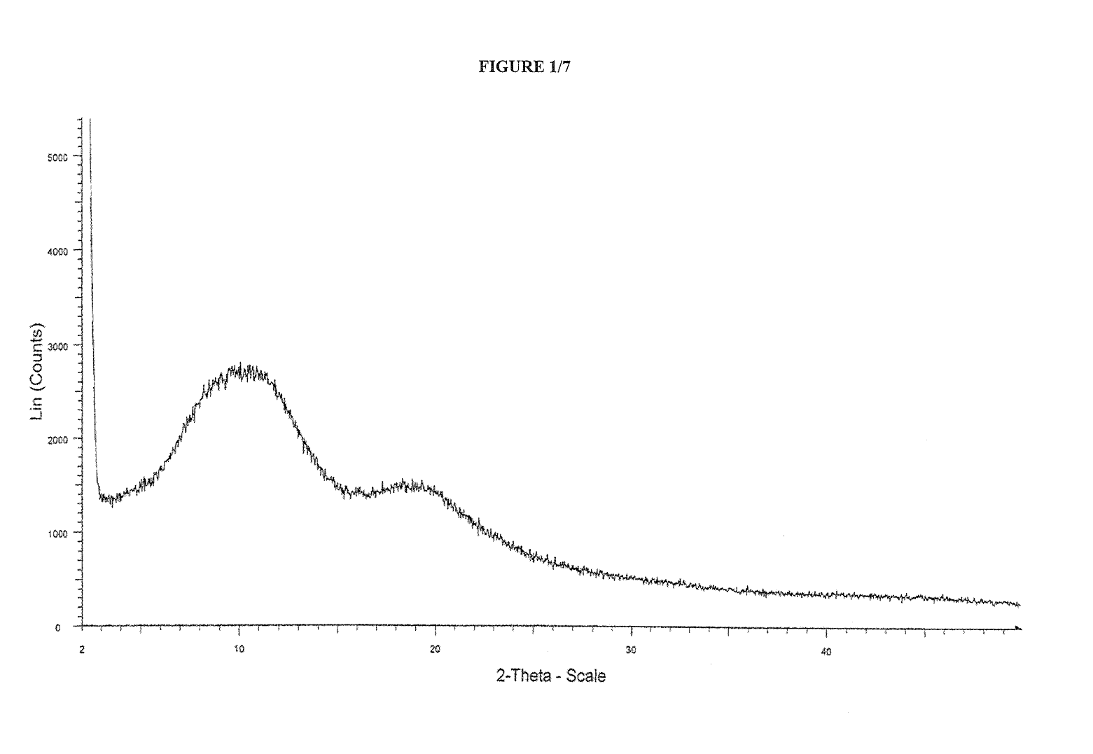

The reaction mixture is optionally filtered and then the solvent is recovered at a temperature above 40° C., till dryness is achieved in the reaction material. This is followed by isolation of amorphous Cabazitaxel. In a preferred embodiment solvent recovery is performed by distillation. In certain observations by inventors of the present application, it was also found that removing the solvent incompletely (i.e. removing at least 60-90% of the total solvent used) followed by adding an anti-solvent having higher boiling point at temperature above 40° C. may also result in amorphous form of Cabazitaxel, which may have composition of amorphous up to at least 95%. The amorphous Cabazitaxel obtained is characterized by X-ray powder diffraction pattern as per FIG. 1 indicating a solid form that lacks the long-range order (a characteristic of crystal) and having no pattern or structure.

The process parameters illustrated above, may be suitably utilized for conversion of various Cabazitaxel solvate forms to amorphous form of Cabazitaxel, employing variations to the process as per requirements. The said solvate form of Cabazitaxel may be selected from diisopropyl ether solvate, ethyl acetate solvate, acetone solvate, ethanol solvate, monohydrate or dihydrate form of Cabazitaxel.

In a preferred embodiment of the present invention solvate form of Cabazitaxel used to prepare amorphous Cabazitaxel is diisopropyl ether solvate form of Cabazitaxel.

In one embodiment of the present application, it provides diisopropyl ether solvate of Cabazitaxel (I),

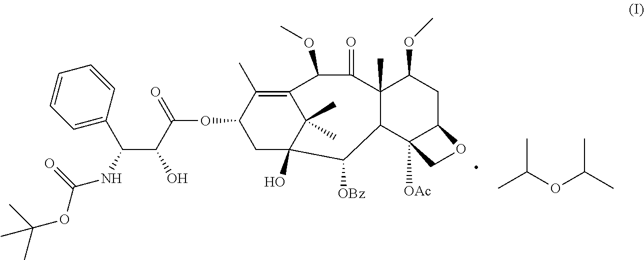

wherein the diisopropyl ether solvate of Cabazitaxel exists as a crystalline form designated as Form-SD, which is characterized by X-ray powder diffraction pattern comprising at least 8 characteristic diffraction angle peaks selected from the XRPD peak set of 7.5, 7.7, 8.6, 13.5, 14.2, 15.0, 17.5, 19.9, 21.7, 21.8, 22.9 and 23.4±0.2 20° and DSC isotherm comprising at least one endothermic peak ranging between 145 to 160° C.

In another embodiment of the present application, diisopropyl ether solvate of Cabazitaxel (I) obtained as crystalline Form-SD is further characterized by IR absorption spectrum having characteristic peaks expressed in cm⁻¹ at approximately 3542 cm⁻¹, 3376 cm⁻¹, 2977 cm⁻¹, 2823 cm⁻¹, 1721 cm⁻¹, 1601 cm⁻¹, 1520 cm⁻¹, 1169 cm⁻¹ and 848 cm⁻¹.

In a further embodiment of the present application, the diisopropyl ether solvate of Cabazitaxel (I) obtained as crystalline Form-SD according to the process of the present invention is characterized by
i. X-ray powder diffraction pattern comprising at least 8 characteristic diffraction angle peaks selected from the XRPD peak set of 7.5, 7.7, 8.6, 13.5, 14.2, 15.0, 17.5, 19.9, 21.7, 21.8, 22.9 and 23.4±0.2 20°.
ii. DSC isotherm comprising the endothermic peak ranging between 145 to 160° C.
iii. IR absorption characteristic peaks at approximately 3542 cm⁻¹, 3376 cm⁻¹, 2977 cm⁻¹, 2823 cm⁻¹, 1721 cm⁻¹, 1601 cm⁻¹, 1520 cm⁻¹, 1169 cm⁻¹ and 848 cm⁻¹.

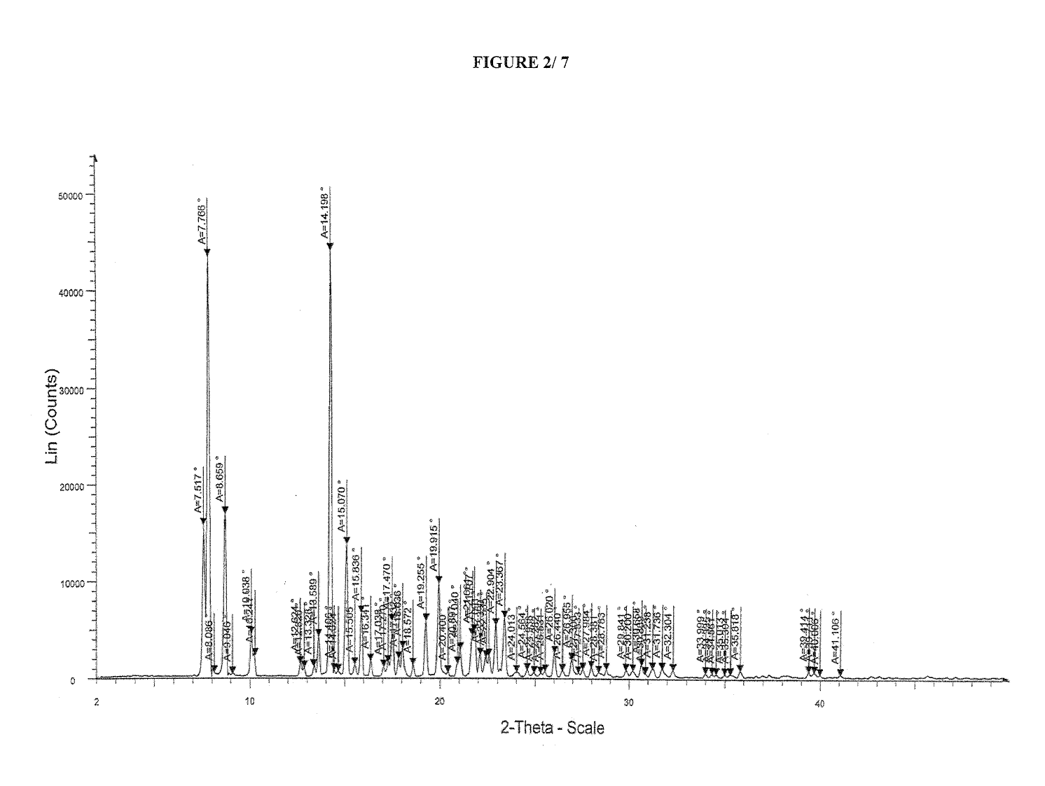

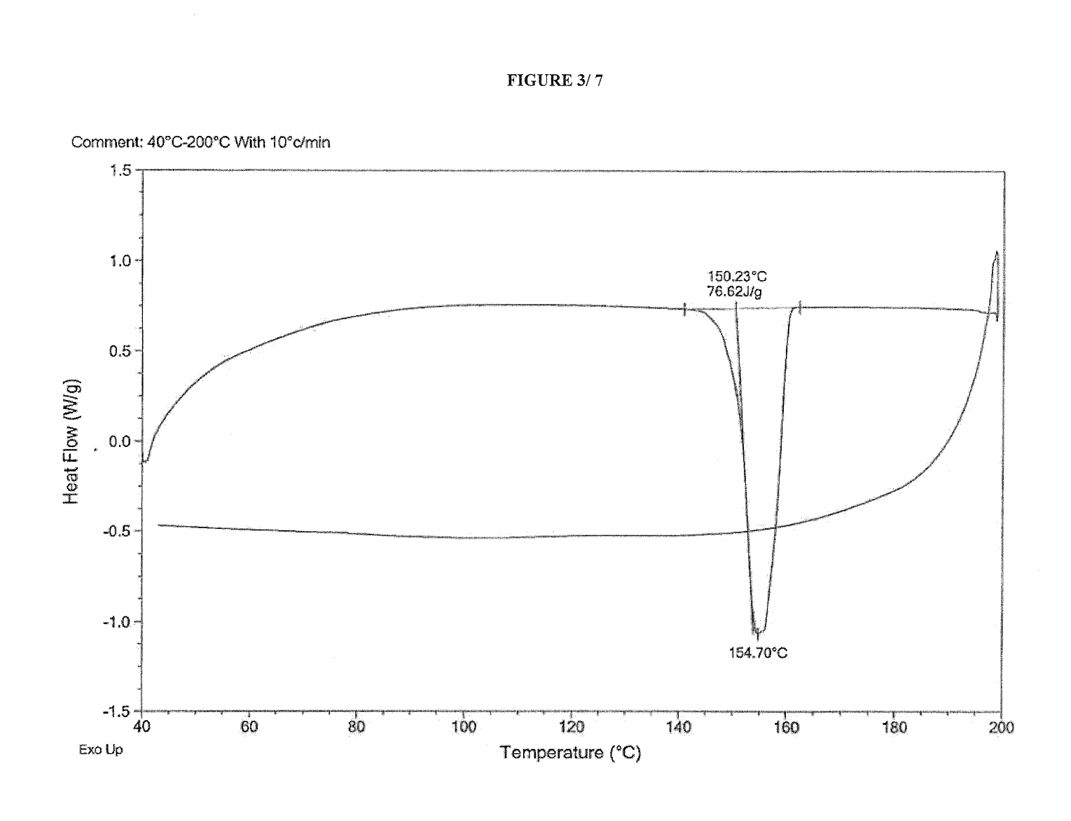

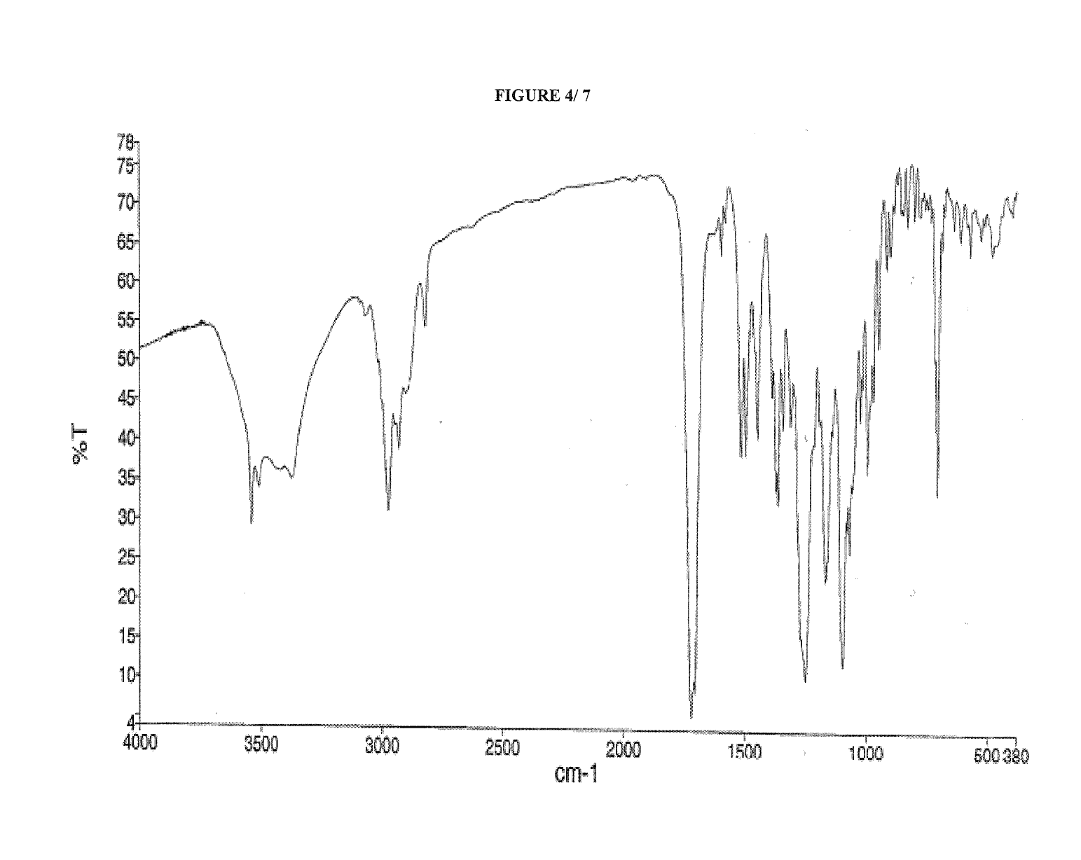

In another embodiment of the present application, substantially pure diisopropyl ether solvate of Cabazitaxel (I) obtained as crystalline Form-SD exhibits an X-ray powder diffraction pattern as shown in FIG. 2, DSC isothermal pattern as shown in FIG. 3 and IR absorption spectrum as shown in FIG. 4. The characteristic peaks and their d-spacing values of the new crystalline Form-SD are tabulated in the Table-1.

TABLE 1
Characteristic XRPD Peaks of Crystalline Form-SD of
Diisopropyl ether solvate of Cabazitaxel (I)

| S. No. | Angle (2θ°) ± 0.20 | d Spacing Value (A°) |
|---|---|---|
| 1. | 7.517 | 11.751 |
| 2. | 7.766 | 11.375 |
| 3. | 8.659 | 10.204 |
| 4. | 10.038 | 8.804 |
| 5. | 13.589 | 6.510 |
| 6. | 14.198 | 6.232 |
| 7. | 15.070 | 5.874 |
| 8. | 15.836 | 5.591 |
| 9. | 17.470 | 5.072 |
| 10. | 18.036 | 4.914 |
| 11. | 19.255 | 4.606 |
| 12. | 19.915 | 4.454 |
| 13. | 21.040 | 4.219 |
| 14. | 21.661 | 4.099 |
| 15. | 21.757 | 4.081 |
| 16. | 22.091 | 4.020 |
| 17. | 22.535 | 3.942 |
| 18. | 22.904 | 3.879 |
| 19. | 23.367 | 3.803 |
| 20. | 26.020 | 3.421 |

Still minor variations in the observed 2 θ° angles values may be expected based on the analyst, the specific XRPD diffractometer employed and the sample preparation technique. Further possible variations may also be expected for the relative peak intensities, which may be largely affected by the non-uniformity of the particle size of the sample. Hence, identification of the exact crystalline form of a compound should be based primarily on observed 2 theta angles with lesser importance attributed to relative peak intensities. The 2 theta diffraction angles and corresponding d-spacing values account for positions of various peaks in the X-ray powder diffraction pattern. D-spacing values are calculated with observed 2 theta angles and copper K a wavelength using the Bragg equation well known to those of having skill in the art of XRPD diffractometry science. In view of possibility of marginal error in the assigning 2 theta angles and d-spacing, the preferred method of comparing X-ray powder diffraction patterns in order to identify a particular crystalline form is to overlay the X-ray powder diffraction pattern of the unknown form over the X-ray powder diffraction pattern of a known form. For example, one skilled in the art can overlay an X-ray powder diffraction pattern of an unidentified crystalline form of Cabazitaxel over FIG. 2 and readily determine whether the X-ray diffraction pattern of the unidentified form is substantially the same as the X-ray powder diffraction pattern of the crystalline Form-SD of this invention. If the X-ray powder diffraction pattern is substantially the same as FIG. 2, the previously unknown crystalline form of Cabazitaxel can be readily and accurately identified as the crystalline Form-SD of this invention.

The new stable crystalline form of Cabazitaxel designated as Form-SD which is characterized by X-ray powder diffraction pattern as shown in FIG-2, DSC isothermal pattern as shown in FIG. 3 and IR absorption spectrum as shown in FIG. 4, is diisopropyl ether Solvate of Cabazitaxel (I), wherein diisopropyl ether content ranges from 8-12% w/w. As an illustrative example, 10% w/w diisopropyl ether content shall be construed as 10 g of diisopropyl ether in 100 g of final API i.e. diisopropyl ether Solvate of Cabazitaxel (I). The diisopropyl ether content in the final API form can be estimated by analytical techniques well known in the art for e.g. by TGA, Gas Chromatography etc. Diisopropyl ether Solvate of Cabazitaxel (I) has been found to be quite stable and easy to handle and store for longer time without any measurable change in its morphology and physicochemical characteristics, while retaining its characteristics within the defined limits. This offers advantages for large scale manufacturing in terms of handling, storage, shelf life and favorable impurity profile.

In another embodiment of the present application, it provides process for preparing diisopropyl ether Solvate of Cabazitaxel (Form-SD), comprising the steps of:
a) Providing a solution of Cabazitaxel in an organic solvent;
b) Adding diisopropyl ether to the reaction mixture;
c) Subjecting the reaction mixture to continuous stirring at temperature ranging between 25 to 55° C.;
d) Filtering the solid material from reaction mass and washing it with diisopropyl ether;
e) Optionally repeating the steps a) to d);
f) Isolating crystalline diisopropyl ether Solvate of Cabazitaxel (Form-SD).

The individual steps of the process according to the present invention for preparing diisopropyl ether Solvate of Cabazitaxel (Form-SD) are detailed separately herein below:

Step a) comprises providing a solution of Cabazitaxel in an organic solvent;

Solution of Cabazitaxel is provided in an organic solvent at an ambient temperature of ˜25° C. Cabazitaxel used as starting material in this reaction may be of any form or purity level, solvated or non-solvated, crystalline or amorphous and may be obtained from any source/method known in the prior art.

Organic solvent used this reaction may be selected from C1-C4 alcohol, DMSO or DMF. Amount of organic solvent used to provide the solution of Cabazitaxel ranges from 2 to 8 times in volume (in mL) w.r.t. the amount of Cabazitaxel used (in g). The solution of Cabazitaxel in the organic solvent is obtained by stirring for time duration ranging from 5 mins to 1 hr depending upon the dissolution obtained. Optionally the temperature of the reaction mixture may be increased to a temperature above 35° C. to somewhat concentrate the reaction mixture, by distillation.
Step b) comprises adding diisopropyl ether to the reaction mixture;

To the reaction mixture obtained in step a) addition of diisopropyl ether is performed. Amount of diisopropyl ether added to the reaction mixture may vary from 2 to 40 times in volume (in mL) w.r.t. the amount of Cabazitaxel initially used (in g). Addition of diisopropyl ether to the reaction mixture may be performed at ambient temperature of ˜25° C. or at a raised temperature of more than 35° C.

Addition of diisopropyl ether to the reaction mixture may be performed in a single lot or in more no. of batches. Rate of addition of diisopropyl ether to the reaction mixture may be controlled as per requirement to achieve final end product characteristics. In a preferred embodiment addition of diisopropyl ether to the reaction mixture was performed slowly over a period of 40 mins.

Step c) comprises subjecting the reaction mixture to continuous stirring at temperature ranging between 25 to 55° C.;

The reaction mixture obtained in step b) is subjected to continuous stirring for a period of 30 mins to 2 hrs. Temperature of the reaction mixture while being stirred may be maintained at an ambient temperature of ˜25° C. or at a raised temperature of up to 55° C. Speed of stirring of the reaction mixture may be controlled as per requirement to achieve final end product characteristics.

When the reaction mass is stirred at temperature of more than 35° C., after completion of the reaction as is checked intermittently on regular intervals, the reaction mixture is allowed to cool to an ambient temperature of ˜25° C. Cooling is performed slowly and may span over time duration of 45 mins to 2 hrs. The reaction mixture obtained after cooling may optionally be again subjected to continuous stirring for a period of 30 mins to 2 hrs.

Step d) comprises filtering the solid material from reaction mass and washing it with diisopropyl ether

Solid material obtained in the reaction mass achieved in step c) is filtered. Filtration may be performed by using any conventional method which is known to the person skilled in art. The solid material obtained after filtration is washed with diisopropyl ether. Optionally washing of the filtered solid material by diisopropyl ether, may be preceded by another washing with mixture of diisopropyl ether and an organic solvent. Organic solvent used in this reaction may be same or different from the organic solvent used in step a).

Step e) comprises optionally repeating the steps a) to d)

The reaction steps a) to d) may be optionally repeated so as to achieve the final end product characteristics and purity levels. Thus, the solid Cabazitaxel material obtained in step d) may be re-dissolved to get a solution of Cabazitaxel in an organic solvent, followed by raising the temperature of the reaction mixture above 35° C., slowly adding diisopropyl ether to the reaction mixture, subjecting the reaction mixture to continuous stirring; cooling of the reaction mixture and again filtering the solid material from reaction mass and washing it with diisopropyl ether. In view of maintaining the equilibrium to the impurity profile compliance, the process may require in-process quality checks to avoid unnecessary repetitions of the same process steps.

Step f) comprises isolating crystalline diisopropyl ether Solvate of Cabazitaxel (I).

The solid material obtained from step d) or e) is subjected to drying for a time duration ranging between 30 mins to 6 hrs. Drying may be performed at RT or at a raised temperature of 35° C. or above. Other parameters of drying may be suitably controlled for e.g. if required drying may also be carried out under reduced pressure conditions which may be suitably utilized by a person skilled in the art.

Process of isolating diisopropyl ether Solvate of Cabazitaxel (Form-SD) comprise processes but not limited to conventional processes including scrapping, if required filtering from slurry, use of anti-solvents, and optional drying which may be carried out at room temperature for the suitable durations.

The end-product obtained after drying in step f) is directly obtained as diisopropyl ether solvate of Cabazitaxel (I) in the form of a crystalline material. This crystalline diisopropyl ether solvate of Cabazitaxel (I) is designated as Form-SD, which is having purity of greater than 99% by HPLC. The process related impurities, including unreacted intermediates, side products, degradation products and other medium dependent impurities, that appear in the impurity profile of the Cabazitaxel may be substantially removed by the process of the present invention resulting in the formation of pure crystalline Cabazitaxel diisopropyl ether solvate (Form-SD).

Diisopropyl ether solvate of Cabazitaxel (I) is found to be a very stable crystal lattice which is adequately stable to handle and store for longer time without any significant or measurable change in its morphology and physicochemical characteristics. This stable form thus, offers various advantages in terms of storage, shelf life and favorable impurity profile. Also the process of the present invention is industrially or commercially suitable and amenable for up scaling and formulation development.

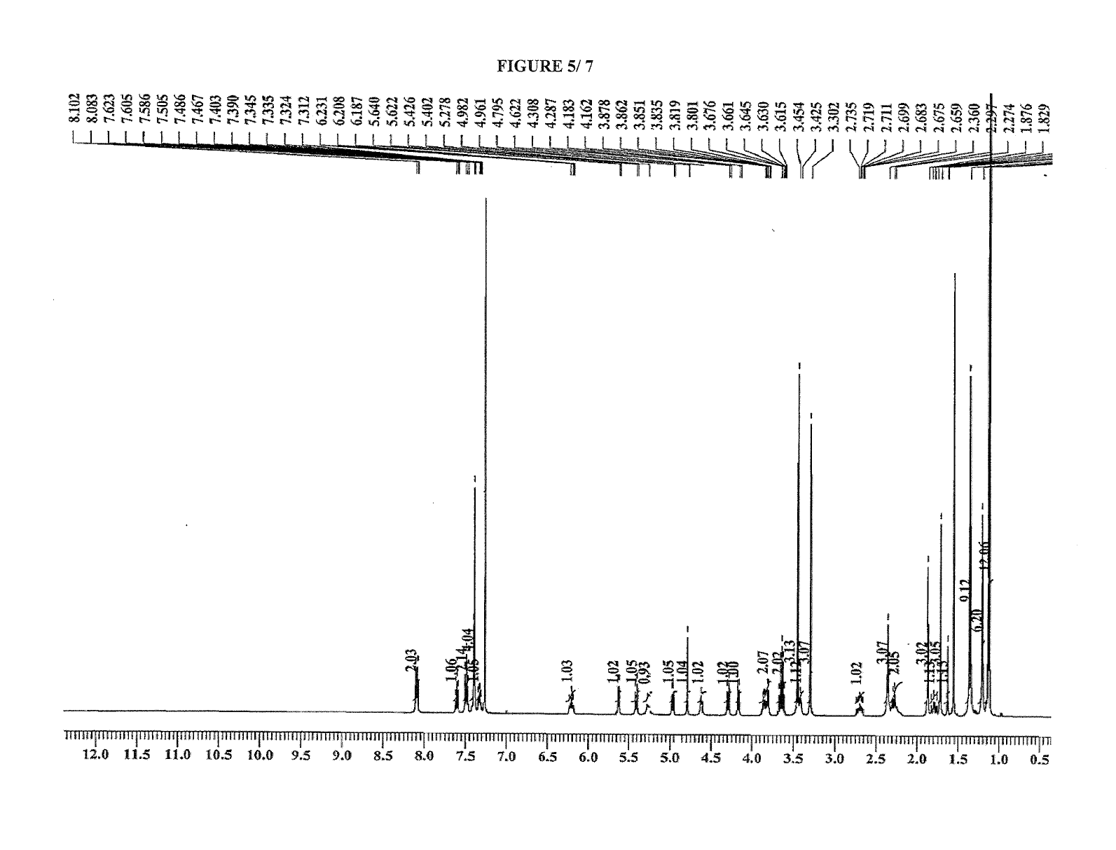

The amorphous form of Cabazitaxel and crystalline Form-SD of Cabazitaxel described herein may be characterized by X-ray powder diffraction pattern (XRPD) and Thermal techniques such as differential scanning calorimetry (DSC) analysis. The said samples of Cabazitaxel were analyzed by XRPD on a Bruker AXS D8 Advance Diffractometer using X-ray source - Cu Ka radiation using the wavelength 1.5418 A and lynx Eye detector. DSC was done on a Perkin Elmer Pyris 7.0 instrument. Illustrative examples of analytical data for the amorphous form of Cabazitaxel obtained from a solvate form of Cabazitaxel and the diisopropyl ether Solvate of Cabazitaxel (Form-SD) are set forth in the FIGS. 1-7.

In a further embodiment according to the specification, the invention also relates to a composition containing diisopropyl ether Solvate of Cabazitaxel (I) or amorphous Cabazitaxel. In yet another embodiment of the invention, the composition may be substantially free of any other known forms of Cabazitaxel. Composition containing diisopropyl ether Solvate of Cabazitaxel (I) or amorphous Cabazitaxel may offer a better pharmacological profile as compared to the prior art known forms.

The diisopropyl ether Solvate of Cabazitaxel (I) or amorphous Cabazitaxel obtained by the process of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerin, propylene glycol or liquid paraffin. In one embodiment of the present invention, it also includes premix comprising one or more pharmaceutically acceptable excipients in the range of 1 to 50% w/w with diisopropyl ether Solvate of Cabazitaxel (I) or amorphous Cabazitaxel, while retaining the nature of the premix.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising diisopropyl ether Solvate of Cabazitaxel (I) or amorphous Cabazitaxel of the present application include, but are but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions of diisopropyl ether solvate of Cabazitaxel (I) or amorphous Cabazitaxel of the present application may also comprise to include the pharmaceutically acceptable carrier used for the preparation of solid dispersion, wherever utilized in the desired dosage form preparation.

Certain specific aspects and embodiments of the present application will be explained in more detail with reference to the following examples, which are provided by way of illustration only and should not be construed as limiting the scope of the invention in any manner.

### EXAMPLE

### Reference Example-A

### Process for preparation of Cabazitaxel

### STEP-A: Preparation of 2′ triethylsilyl protected Cabazitaxel

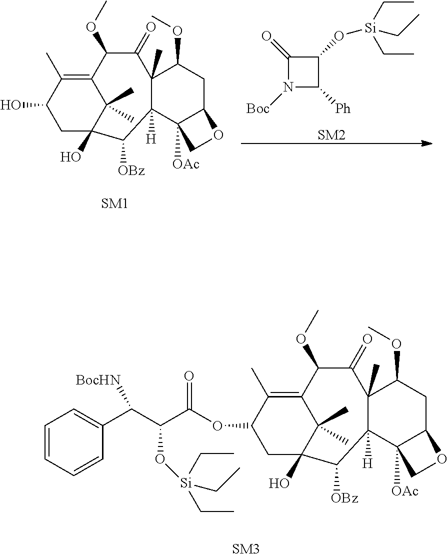

A solution of Lithium bis(trimethylsilyl) amide (LiHMDS) (0.9 M/THF, 11.7 ml, 10.4 mmol) was added drop wise over 5 min to a stirred suspension of 7,10-Dimethoxy 10-DAB-III (SM1) (5.0 g, 8.7 mmol) in THF (125 ml) at −10 to −5° C. under nitrogen atmosphere. The reaction mixture was stirred for 5-10 min at −10 to −5° C. under nitrogen atmosphere. Added triethylsilyl protected lactam (SM2) (5.0 g, 13.1 mmol) to the reaction mixture at −10 to −5° C. over a period of 5 min. The reaction mixture temperature was raised to 20-25° C. and stirred for lh. The reaction mixture was cooled to 10-15° C., quenched with saturated ammonium chloride (100 ml) and extracted with ethyl acetate (100 ml). The organic layer was washed with water followed by saturated sodium chloride solution. The organic layer was dried over sodium sulphate and concentrated to yield crude triethylsilyl protected Cabazitaxel (SM3) (7.5 g, Yield: 90%, Purity: 85.59%). The crude triethylsilyl protected Cabazitaxel was further purified by column chromatography over silica gel (100-200 mesh) using ethyl acetate and hexane to get a product (5.2 g, Yield: 62.6%, Purity: 94.97%).

### STEP-B: Purification of 2′ triethylsilyl protected Cabazitaxel

To the triethylsilyl protected Cabazitaxel (3.2 g) obtained above was added 8.64 ml of ethyl acetate. The resulting suspension was stirred for 10 min at 60-65° C. The reaction mass was cooled to 45-50° C. and 17.28 ml of hexane was added drop wise over a period of 30 min. The resulting slurry was cooled to 20-25° C. over a period of 1 h and stirred for 30 min. The solid was filtered and washed with 6.4 ml of hexane and dried to obtain triethylsilyl protected Cabazitaxel (1.8 g, Yield: 56.0%, Purity: 99.46%).

### STEP-C: Deprotection of 2′ triethylsilyl protected Cabazitaxel

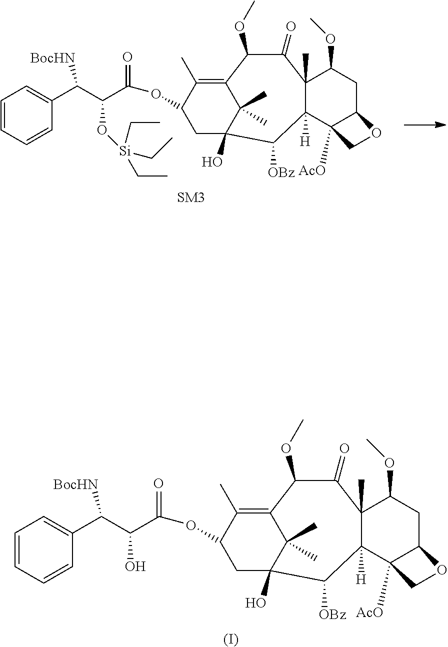

In a solution of tetra-n-butyl ammonium fluoride (1 M/THF, 3.2 ml, 3.1 mmol) was added a solution of 2′ triethylsilyl protected Cabazitaxel (2.5 g, 2.6 mmol) in THF (50.0 ml) at 0-5° C. under nitrogen atmosphere. The resulting solution was stirred for 1 hr at 0-5° C. The reaction mixture was diluted with ethyl acetate (50.0 ml) and quenched with saturated ammonium chloride solution (50.0 ml) at 0-5° C. The organic layer was separated and washed with water (2x50.0 ml) followed by saturated sodium chloride solution (50.0 m1). The organic layer was dried over anhydrous sodium sulphate and concentrated to yield 2 g of crude cabazitaxel.

### STEP-D: Purifying the crude Cabazitaxel

Crude Cabazitaxel (2 gm) was dissolved in ethyl acetate (50mL) and heated to 60-65° C. and stirred for 10 min. The ethyl acetate was distilled up to 8 mL and the suspension was stirred for 1h at room temperature and filtered. The filter bed was washed with 30% ethyl acetate in hexane (10 ml) and dried to obtain pure cabazitaxel (1.56 g, Yield: 72.0%, Purity: 99.48%).

### Reference Example-B

### Process for Preparation of Cabazitaxel

Cabazitaxel used as starting material in the invention of the present application, can also be synthesized according to the process mentioned in US 6,331,635 B1 which has been incorporated herein by way of reference. The process mentioned in U.S. Pat. No. 6,331,635 B1 is summarized in the following scheme:

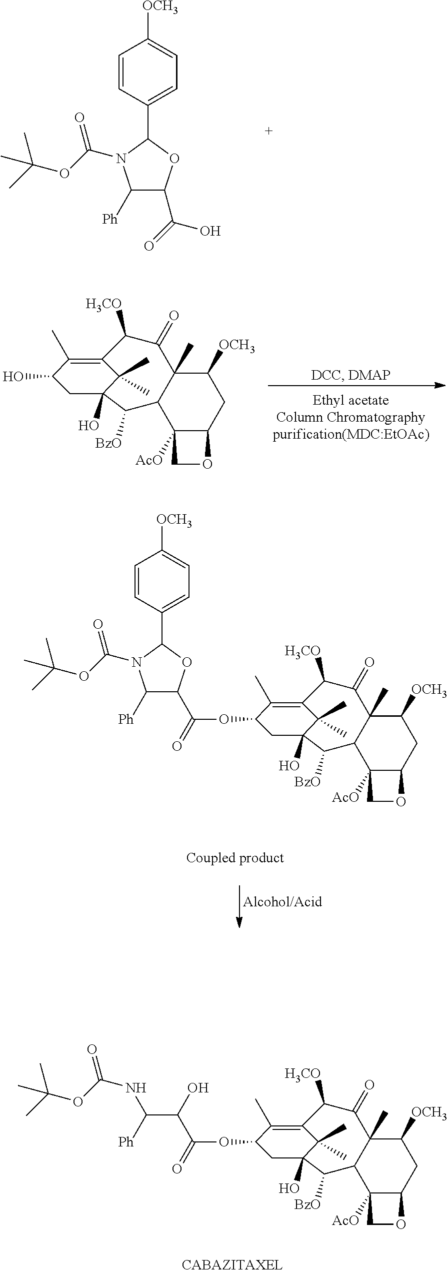

### Example-01

### Preparation of Cabazitaxel diisopropyl ether Solvate (I) i.e. Form-SD

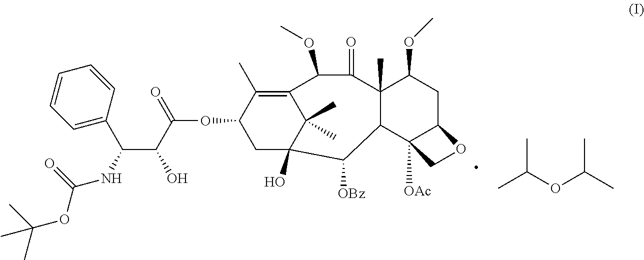

2.0 g Cabazitaxel in 4.0 mL methanol was charged in to a 100 mL round bottom flask (RBF) at ˜25° C. The reaction mixture was stirred for 10 mins at this temperature, followed by addition of 40.0 mL diisopropylether. The resulting slurry was stirred for 1 hr. The solid obtained was filtered and washed with 4.0 mL diisopropylether. Then the solid material obtained was suck dried for 30 mins.

The solid material obtained above was re-transferred into 100 mL RBF containing 4.0 mL methanol at ˜25° C. Then the temperature of reaction mixture to was raised to about 45° C., wherein stirring was performed for 10 mins while keeping the temperature constant. Stirring was followed by slow addition of 12.0 mL diisopropylether to the reaction mixture over a period of 35 mins. After the completion of diisopropylether addition, the reaction mixture was stirred for 1 hr keeping the temperature constant. Then the reaction mixture was slowly cooled to ˜30° C. /over a period of 90 mins. The cooled reaction mixture was further stirred for 1 hr.

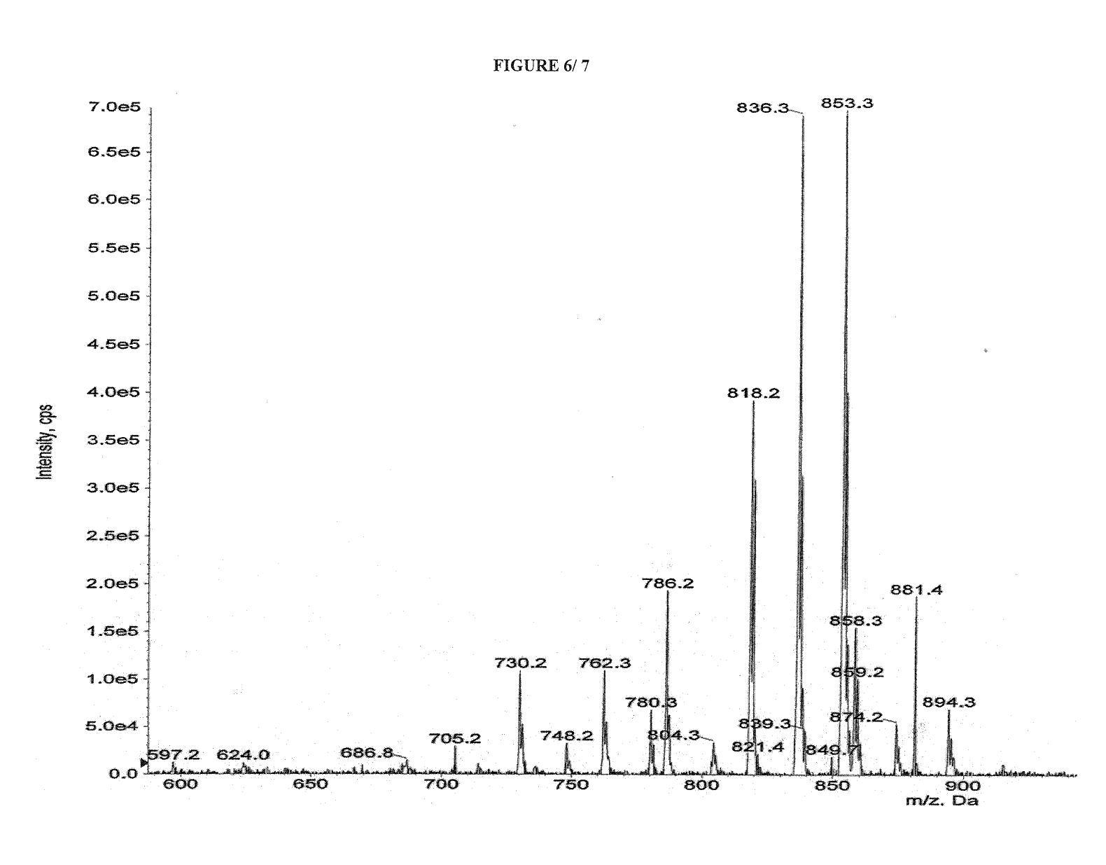

The solid material obtained in the above reaction mass was filtered and the wet cake was washed with 2.0 mL, 5% Methanol-diisopropylether solution. Another set of washing was given to the obtained solid material with 4.0 mL diisopropyl ether. Then the solid material was suck dried for 30 mins. Further drying was carried out at 45° C. under reduced pressure for 3 hrs. The dried material was then unloaded to obtain Cabazitaxel diisopropyl ether solvate (1.24 g) having M. Pt: 154.7° C. (by DSC); Purity: 99.36% (By HPLC); Mass spectrum as per FIG. 6

¹H NMR (400 MHz, CDCl₃) δ 8.08-8.10 (d, 2H), 7.58-7.62 (t, 1H), 7.46-7.52 (dd, 2H), 7.31-7.40 (m,5H), 6.18-6.23 (t, 1H), 5.62-5.64 (d, 1H), 5.40-5.42 (d, 1H), 5.27 (broad doublet, 1H), 4.96-4.98 (broad doublet, 1H), 4.79 (s, 111), 4.62 (broad singlet, 1H), 4.16-4.30 (dd, 2H), 3.83-3.87 (dd, 111), 3.80-3.81 (d, 1H), 3.61-3.67 (septet, 2H), 3.42 (broad singlet, 1H), 3.30 (s, 2H), 3.45 (s, 2H), 2.36 (s, 3H), 2.27-2.29 (m, 2H), 1.87 (s, 3H), 1.71 (s, 3H), 1.79 and 2.69 (m, 2H), 1.64 (s, 1H), 1.35 (s, 9H), 1.20-1.21 (s, 6H), 1.12-1.13 (d, 12H)

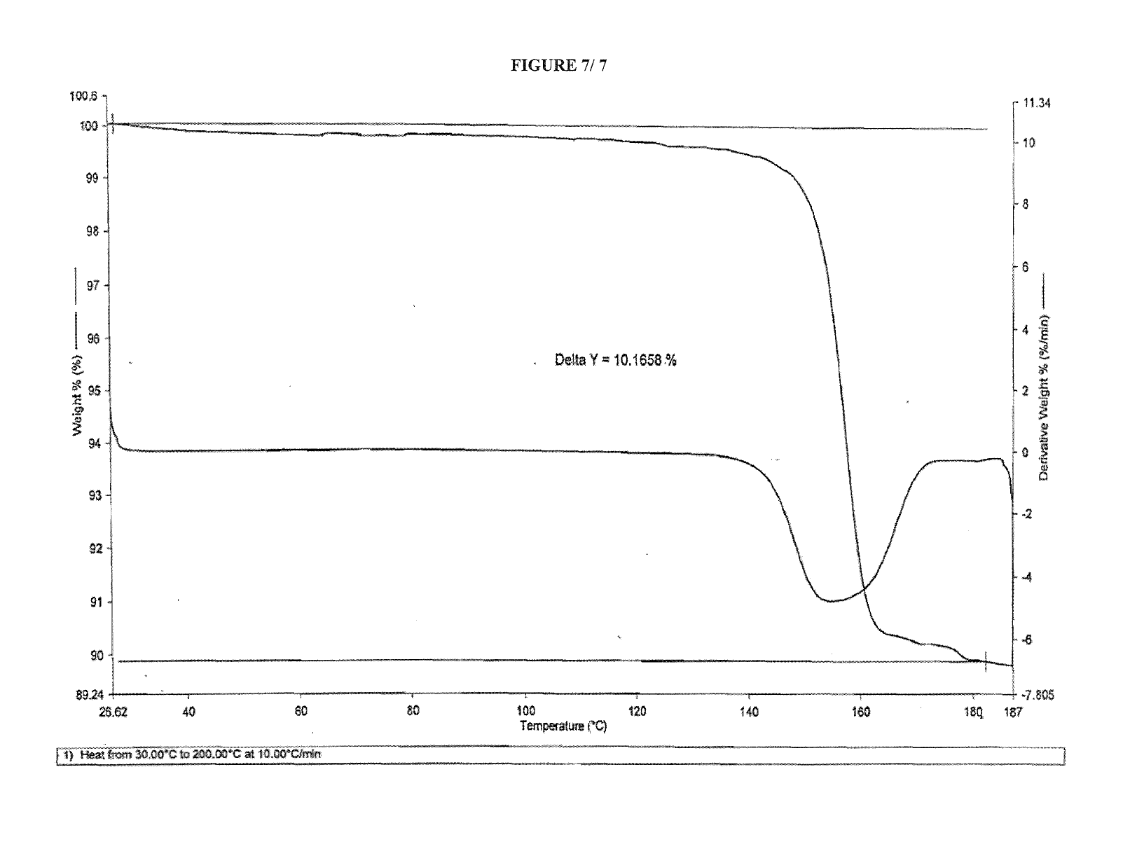

Cabazitaxel diisopropyl ether solvate (I) is obtained as crystalline Form-SD having XRPD pattern as per FIG. 2; IR spectrum as per FIG. 4; and TGA spectrum as per FIG. 7.

### Example-02

### Preparation of Cabazitaxel diisopropyl ether Solvate (Crystalline Form-SD)

2.5 g Cabazitaxel in 5.0 mL methanol was charged in to a 100 mL round bottom flask (RBF) at ˜30° C. The reaction mixture was stirred for 10 mins at this temperature, followed by addition of 50.0 mL diisopropylether. The resulting slurry was stirred for 1 hr. The solid obtained was filtered and washed with 5.0 mL diisopropylether. Then the solid material obtained was suck dried for 30 mins.

The solid material obtained above was again transferred into 100 mL RBF containing 5.0 mL methanol at ˜30° C. Then the temperature of reaction mixture to was raised to about 45° C., wherein stirring was performed for 15 mins while keeping the temperature constant. Stirring was followed by slow addition of 15.0 mL diisopropylether to the reaction mixture over a period of 40 mins. After the completion of diisopropylether addition, the reaction mixture was stirred for 1 hr keeping the temperature constant. Then the reaction mixture was slowly cooled to ˜25° C. over a period of 80 mins. The cooled reaction mixture was further stirred for 1 hr.

The solid material obtained in the above reaction mass was filtered and the wet cake was washed with 2.5 mL, 5% Methanol-diisopropylether solution. Another set of washing was given to the said solid material by 5.0 mL diisopropyl ether. Then the solid material obtained was suck dried for 30 mins. Further drying of the solid material was carried out at 50° C. under reduced pressure for 3 hrs. The dried material was then unloaded to obtain Cabazitaxel diisopropyl ether solvate, Form-SD (1.57 g) having M. Pt: 153.22° C. (by DSC) and Purity: 99.19% (By HPLC).

XRPD pattern is similar to FIG. 2; IR spectrum is similar to FIG. 4; and TGA spectrum is similar to FIG. 7.

### Example-03

### Preparation of Cabazitaxel diisopropyl ether Solvate (Crystalline Form-SD)

In to a 250 mL RBF, 10.0 g Cabazitaxel was charged with 40.0 mL methanol at 25-30° C. The reaction mixture was stirred for 10 mins at 25-30° C. and methanol was distilled out up to 20.0 mL at 40-45° C. under reduced pressure. To the concentrated solution, 60.0 mL Diisopropylether was added over a period of ˜30 mins. The resulting slurry was stirred for another 45 mins and then over a period of 30 mins the reaction temperature was cooled to ˜25° C. The cooled reaction mixture was stirred for 1 hr and then filtered followed by washing with 20.0 mL Diisopropylether. The wet material obtained was then suck dried for 30 mins, to obtain Cabazitaxel diisopropyl ether solvate, Form-SD (8.1 g) having Purity: 99.84% (By HPLC).

### Example-04

### Preparation of Cabazitaxel ethyl acetate Solvate (1:1)

To the compound obtained above in Reference example-01 (1.5 g) was added ethyl acetate (7.5 ml) at 25-30° C. and the suspension was stirred for 6 hrs at 25-30° C. The solid was filtered and washed with 30% ethyl acetate in hexane (3.0 ml) an dried under vacuum at 40-45° C. for 6 h to yield Cabazitaxel ethyl acetate solvate (1.4 g) having M. Pt. of 159-162° C.

### Example-05

### Preparation of Cabazitaxel acetone Solvate

In a RB flask equipped with magnetic stirrer, and thermometer, Cabazitaxel ethyl acetate solvate (2.0 g) was dissolved in acetone (10 ml×2) at 25-30° C. and acetone was distilled up to 1.0 vol at 45-50° C. (Bath temperature) under reduced pressure. The reaction mass was cooled, diluted with acetone (18 ml) and stirred for 10 min at 25-30° C. DM water (10.0 ml) was slowly added to the above mass over a period of 30 min at 25-30° C., followed by seeding with Cabazitaxel acetone solvate (20 mg) and the resulting suspension was stirred for 22 hrs at 25-30° C. DM water (15.0 ml) was slowly added over a period of 2-3 hrs at 25-30° C. and stirred for 2 hrs. The solid was vacuum filtered and washed with acetone water mixture (10.0 ml i.e. acetone-4.5 ml and water-5.5 ml) to yield Cabazitaxel acetone solvate having melting point of 158-164° C. (1.8 g, Yield: 90.0%, HPLC Purity: 99.95%).

### Example 06

### Preparation of amorphous Cabazitaxel from Cabazitaxel diisopropyl ether Solvate (Form-SD)

In a RB flask equipped with magnetic stirrer, thermometer and a gas bubbling tube was added Cabazitaxel diisopropyl ether solvate (220 mg) and 18 ml of methanol at room temperature. The reaction mass was stirred to dissolve completely at 30° C. for 20 minutes. The temperature of the clear solution was raised up to 55° C. The temperature was maintained for 30 mins and the solvent was recovered at this temperature up to dryness to afford amorphous Cabazitaxel. Yield: 160 mg

### Example 07

### Preparation of Amorphous Cabazitaxel from Cabazitaxel diisopropyl ether Solvate (Form-SD)

In to a 1 L single RBF, 10.0 g Cabazitaxel diisopropyl ether solvate (Form-SD) was charged along with 400.0 mL methanol at ˜25° C. The reaction mixture was stirred for 10 mins and then filtered through 0.2p, filter paper. From the filtered reaction mixture, methanol was distilled up to dryness at ˜50° C. under reduced pressure. The material obtained was unloaded and further dried at ˜45° C. under reduced pressure for 4 firs, to afford amorphous Cabazitaxel (8.0 g) having Purity: 99.80% (By HPLC).

### Example 08

### Preparation of amorphous Cabazitaxel from Cabazitaxel Ethyl acetate Solvate

In a RB flask equipped with magnetic stirrer, thermometer and a gas bubbling tube was added Cabazitaxel ethyl acetate solvate (200 mg) and 16 ml of methanol at room temperature. Stir the reaction mass to dissolve completely at 25-30° C. for 15-20 minutes. Raise the temperature of the clear solution up to 50-55° C. Maintain the temperature for 30 min and recover the solvent at this temperature up to dryness to afford amorphous material. Yield: 150 mg

### Example 09

### Preparation of amorphous Cabazitaxel from Cabazitaxel acetone Solvate

In a RB flask was equipped with magnetic stirrer and thermometer, Cabazitaxel acetone solvate (500 mg) was dissolved in methanol (40.0 ml) and stirred at 25-30° C. for 15-20 min. The above clear solution was filtered and the temperature was raised to 50-55° C. The reaction mass temperature was maintained for 30 min and the solvent was recovered at this temperature up to dryness to afford amorphous material.

While the foregoing provides a detailed description of the preferred embodiments of the invention, it is to be understood that the descriptions are illustrative only of the principles of the invention and not limiting. Furthermore, as many changes can be made to the invention without departing from the scope of the invention, it is intended that all material contained herein be interpreted as illustrative of the invention and not in a limiting sense.

We claim:

1) A process for preparing Cabazitaxel from the solvate form of Cabazitaxel, comprising the steps of
i. providing a solution of Cabazitaxel solvate form with solvent methanol or 2-methoxyethanol;
ii. maintaining the reaction mass under stirring to dissolve completely at temperature ranging from 10-35° C.;
iii. heating the solution up to the temperature ranging from 40-60° C.;
iv. optionally holding the solution under stirring for at least 20 minutes but not more than 60 minutes; and
v. removing the solvent by distillation and isolating the amorphous Cabazitaxel;

2) A process for preparing Cabazitaxel according to claim 1, wherein Cabazitaxel solvate is diisopropyl ether solvate (I), characterized by X-ray powder diffraction pattern comprising at least 8 characteristic diffraction angle peaks selected from the XRPD peak set of 7.5, 7.7, 8.6, 13.5, 14.2, 15.0, 17.5, 19.9, 21.7, 21.8, 22.9 and 23.4+0.2 2θ° and DSC isotherm comprising at least one endothermic peak ranging between 145 to 160° C.

3) A process for preparing Cabazitaxel according to claim 1, wherein cabazitaxel solvate is Diisopropyl ether solvate of Cabazitaxel (I), 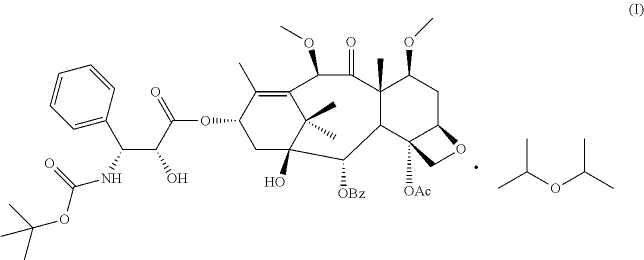
characterized by X-ray powder diffraction pattern comprising at least 8 characteristic diffraction angle peaks selected from the XRPD peak set of 7.5, 7.7, 8.6, 13.5, 14.2, 15.0, 17.5, 19.9, 21.7, 21.8, 22.9 and 23.4±0.2 2θ° and DSC isotherm comprising at least one endothermic peak ranging between 145 to 160° C.

4) A process for preparing Cabazitaxel according to claim 1, wherein cabazitaxel solvate is Diisopropyl ether solvate of Cabazitaxel (I), characterized by diisopropyl ether content ranging from 8-12% w/w.

5) A process for preparing Cabazitaxel according to claim 1, wherein in step i. of providing solution of Cabazitaxel solvate is carried out at temperature ranging between 20-30° C. in solvent selected from methanol or 2-methoxyethanol.

6) A process for preparing Cabazitaxel, comprising the steps of:
a) providing a solution of Cabazitaxel in an organic solvent;
b) adding diisopropyl ether to the reaction mixture;
c) subjecting the reaction mixture to continuous stirring at temperature ranging between 25 to 55° C.;
d) filtering the solid material from reaction mass and washing it with diisopropyl ether;
e) optionally repeating the steps a) to d);
f) isolating crystalline diisopropyl ether solvate of Cabazitaxel (I) characterized by X-ray powder diffraction pattern comprising at least 8 characteristic diffraction angle peaks selected from the XRPD peak set of 7.5, 7.7, 8.6, 13.5, 14.2, 15.0, 17.5, 19.9, 21.7, 21.8, 22.9 and 23.4±0.2 2θ° and DSC isotherm having at least one endothermic peak ranging between 145 to 160° C.; and
g) converting diisopropyl ether solvate of Cabazitaxel (I) obtained in step-f) to amorphous Cabazitaxel.

7) A process for preparing Cabazitaxel according to claim 6, wherein the organic solvent used in step a) is selected from C₁-C₄ alcohol, DMSO or DMF.

8) A process for preparing Cabazitaxel according to claim 6, wherein in step a) the solution of Cabazitaxel in an organic solvent is optionally heated to a temperature above 35° C.

9) A pharmaceutical composition comprising Cabazitaxel obtained by the process according to claim 6, with at least one pharmaceutically acceptable excipient.
